(19) United States
(12) Patent Application Publication
(10) Pub. No.: US 2010/0256620 A1
(43) Pub. Date: Oct. 7, 2010

(54) Thin flexible cryoprobe operated by krypton

(75) Inventor: Ben-Zion Maytal, Atlit (IL)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: Galil Medical Ltd., Yokneam (IL)

(21) Appl. No.: 11/651,997

(22) Filed: Jan. 11, 2007

Related U.S. Application Data
(60) Provisional application No. 60/758,215, filed on Jan. 12, 2006.

(51) Int. Cl. A61B 18/02

(52) U.S. Cl. 606/21; 606/22

(57) ABSTRACT

The present invention relates to Joule-Thomson cooling systems operable to achieve rapid-response extreme cooling utilizing krypton as a cooling gas. Devices presented do not require heat exchangers between gas input and output conduits, and consequently are characterized by rapid response, small diameters and enhanced flexibility.

### RELATED APPLICATIONS

This application claims the benefit of U.S. Provisional Patent Application No. 60/758,215 filed Jan. 12, 2006, the contents of which are hereby incorporated by reference.

### FIELD AND BACKGROUND OF THE INVENTION

The present invention relates to an ultra-thin and ultra-flexible cryoprobe, and more particularly to a cryoprobe operated by krypton and able to achieve cryoablation temperatures without requiring a counter-flow heat exchanger. Construction of the probe absent a heat exchanger enables a high degree of miniaturization and enhances flexibility.

Some cryoprobes cool by evaporation of a liquefied gas supplied to the probe and evaporated within a cooling tip of the probe. In contrast, an important category of cryosurgical probes is based on miniature Joule-Thomson cryocoolers. These probes cool by expansion of a high-pressure gas, which expansion cools the gas and may generate a bath of liquid cryogen at the tip of the probe. Such probes absorb heat from the living tissue after being cooled both by gas expansion and by evaporation of liquefied gas which liquefies during that expansion.

Joule-Thomson cryocoolers often operate in an open cycle, wherein the gas expansion process is fed by a high-pressure reservoir and exhausted expanded gas is released to the atmosphere.

Among various types of cryosurgical apparatuses reported, we cite the following representative examples:

U.S. Pat. No. 3,477,434 to Hood Charles B. et al. discloses a cryosurgical apparatus characterized by a housing adapted for manual manipulation by a surgeon and a probe portion provided with a cold tip. The apparatus includes a miniature cryogenic heat exchanger including a warmer path extending axially within the handle portion for conducting a flow of refrigerant to the cold tip and a colder path for conducting a counter flow of the cooled refrigerant away from the tip, with thermal contact between the first and second paths providing a heat-exchanging relationship between them.

U.S. Pat. No. 3,800,552 to Bulat, Thomas J. et al. discloses an apparatus for directly converting a gas into a liquid to lower the temperature of a cryogenic surgical instrument. The apparatus comprises a tube with a linear entrance and exit section helically wound around a core, and a thermal conductive member surrounding the tube between the entrance and exit sections. Gas under pressure is connected to the entrance section of the tube. A sleeve with a closed end frictionally engages and surrounds the thermal conductive member to form a closed chamber adjacent the exit section of the tube. The pressurized gas is throttled upon leaving the exit section causing the temperature in the chamber to be lowered to between −80° to −250° C., causing the gas to liquefy. A path through the thermal conductive member from the chamber to the atmosphere permits thermal energy in the throttled gas to be dissipated to the pressurized gas in the tube. The liquefied gas in the chamber correspondingly cools the closed end of the sleeve allowing the external surface thereof to be used as a cryogenic surgical probe.

U.S. Pat. No. 5,800,487 to Mikus, Paul W. discloses a cryo-cooler and cryoprobes for use in cryosurgery comprising a flow-directing sheath surrounding a heat exchanger, and a cryostat having a high pressure gas supply line supplying a Joule-Thomson nozzle.

U.S. Pat. Nos. 5,522,870 and 5,540,062 to Maytal Ben-Zion disclose cryoprobes comprising heat exchangers for precooling cooling gas directed towards a treatment tip.

As may be seen from the above examples and from similar examples well known in the art, the essential elements of a traditional Joule-Thomson (Linde-Hampson) cryocooler are (a) an expansion orifice through which a high-pressure gas expands into an expansion chamber, and (b) a counter-flow heat exchanger for exchanging heat between warm high-pressure gasses and cold depressurized gasses, for pre-cooling high-pressure cooling gas directed toward the expansion orifice.

The pressure drop at the nozzle (orifice) occurs at constant enthalpy and is accompanied by a temperature drop, also known as the integral adiabatic Joule-Thomson effect, so that, according to a well-known formula,

h(P₁,T₁)=h(P₂,T₂)

T₂−T₁=ΔTₕ

where the outlet pressure is atmospheric pressure,

P₂=0.1 MPa

Coolants are chosen so that T₁ is below the Joule-Thomson inversion point, therefore,

ΔTₕ<0.

Coolants commonly used in prior-art practice, when cooling to below 100 K. is desired, are nitrogen, air and argon with their corresponding normal boiling points of 77.3 K, 78.5 K, and 87.3 K.

It may be noted that in these cases, the highest associated temperature reduction, ΔTₕ which may be produced by expansion from commercially available gas concentrations to atmospheric pressure at room temperature are about, 40 K, 42 K, and 85 K. These temperature differentials are therefore not sufficiently large to obtain liquefaction of the expanding gas.

The counter-flow heat exchanger seen in the above-cited prior art patents and present generally in all prior art cryoprobes using Joule-Thomson cooling to achieve cryoablation temperatures serves to magnify the isenthalpic temperature drop ΔTₕ, lowering the outlet temperature down to the boiling point of the coolant.

In attempting to reduce the diameter of cryoprobes, it is the size of the counter-flow heat exchanger which is generally the limiting parameter determining minimum diameter of the probe. Heat exchangers used in cryoprobes currently known in the art generally comprise a relatively bulky heat-exchanging configuration, usually a tightly coiled tube built to enhance heat exchange by creating a large surface of contact between a gas input tube carrying high-pressure gas and a gas exhaust tube carrying cold expanded gas exhausting from the cold operating tip of the probe. This heat exchanger is usually positioned just before the Joule-Thomson expansion orifice of the operating tip. Coiled gas conduits or similar arrangements are need to enhance thermal flow between high pressure and low pressure gas conduits. Cold expanded gas flows over the coiled tube of the heat exchanger, cooling the high pressure gas before it reaches the expansion orifice. For example, element 22 in FIG. 1 of U.S. Pat. No. 3,477,434 (cited above) defines the size of the probe shown in that figure. As tube manufacturing technology advanced beyond that contemplated by Hood, the attainable minimal diameter of cryoprobes was reduced. However, to this day, necessary size of an included necessary heat exchanger is a main impediment to the manufacture of a thin cryoneedle.

Relating to another aspect of the invention, it is noted that for a variety of cryosurgical applications it is appropriate to create a very small ablation zone. It has been found that for such applications it is preferable to use short cooling times at very low temperatures, rather than long cooling times at relatively higher temperatures. Short cooling times may also particularly be desirable in specific anatomical contexts, such as when cooling or ablation is used inside a blood vessel, since blood flow in the vessel is typically impeded or wholly blocked during the operation. Thus there is a widely felt need for, and it would highly advantageous to have, a cryoprobe operable to provide temperatures lower than those typically provided by cryoprobes known to prior art.

It is another disadvantage of heat exchangers that, in addition to being relatively bulky, they are also typically relatively inflexible. Thus flexibility of prior art cryoprobes is limited by presence of an inflexible heat exchanger. This problem may be partially overcome by positioning a heat exchanger distant from an operating tip (e.g. in a handle), yet that solution tends to limit the effective length of such a probe, since distance between heat exchanger and Joule-Thomson expansion chamber introduces thermal inefficiencies.

Relating to yet another aspect of the invention, it is noted that Joule-Thomson cooling involving a heat exchanger is of necessity a process with a relatively long lead time, as efficient cooling is only achieved after cold expanded gasses have cooled the heat exchanger itself to the point where input gasses are cooled to their optimal input temperature. For certain medical and industrial applications rapid-onset cooling is desirable. For some applications, military infra-red detection and localization of incoming missiles, for example, rapid-onset cooling may be essential. Thus there is a widely felt need for, and it would highly advantageous to have, a Joule-Thomson cooling system operable to achieve low temperatures within a very short lead time.

Some applications of krypton as a coolant for Joule-Thomson cryocooling have been reported. Because of its somewhat elevated boiling point, krypton was employed as a precoolant for a final stage of nitrogen as reported by Pope, A. W., in “Development of a two stage alternate Joule-Thomson cryocooler for AAWS-M. Risk reduction” published by the US Army Missile Command, Technical Report RD-AS-91-22, 1991.

Krypton was also used as the coolant for a cryosurgical probe with a heat exchanger and compared with an argon-operated similar probe, as reported by Longsworth, R. C. in “Considerations in applying open cycle J-T cryostats to cryosurgery”, pp. 783-792, published in Cryocoolers 11, Kluwer Academic/Plenum Publishers, 2001. That publication describes two kinds of cryoprobes of 3.4 mm OD, one with finned tube heat exchanger and the other with a matrix heat exchanger. The induced backpressure was 700 kPa, which means that the temperature of the bath of argon was elevated to 110 K. It means that the temperature of a small size probe of argon comes quite close to the normal boiling point of krypton. Put another way, the process of miniaturization of an argon probe is associated with elevation of the operating temperature, and consequently a reduction in the cooling capacity of the probe.

### SUMMARY OF THE INVENTION

According to one aspect of the present invention there is provided a cryoprobe system comprising: a gas supply operable to supply high-pressure krypton gas; and a flexible gas conduit attachable at a proximate end to the gas supply and having an orifice positioned at a distal end of the conduit, the system being characterized in that when high-pressure krypton gas is supplied by the high-pressure cooling gas supply to the conduit, a mixture of cold, low pressure gas and liquid droplets forms outside the orifice.

According to further features in preferred embodiments of the invention described below, the mixture forms at a temperature inferior to 125 K and the outer diameter of the conduit is less than 1.5 mm.

According to another aspect of the present invention there is provided a cryoprobe system comprising a supply of high-pressure krypton and a cryoprobe which comprises a cooling tip which comprises an expansion chamber and a shaft which comprises a gas exhaust lumen operable to exhaust gas from the cooling tip and a gas input lumen operable to receive high-pressure krypton supplied by the high-pressure krypton supply and having a distal orifice operable to permit passage of krypton from the gas input lumen to the expansion chamber.

According to further features in preferred embodiments of the invention described below, the system does not comprise a portion designed as a heat exchanger serving to facilitate exchange of heat between the gas input lumen and the gas exhaust lumen.

According to still further features in preferred embodiments of the invention described below, the system is operable to form a mixture of cold krypton gas and liquefied krypton droplets when krypton supplied by the gas supply traverses the orifice and enters the expansion chamber.

The gas input lumen may be positioned within the gas exhaust lumen. Preferably length of the gas input lumen differs from length of the gas exhaust lumen by less than 5%.

In a preferred embodiment heat conductance between the gas input lumen and the gas exhaust lumen per unit length along a subsection of the shaft differs by not more than 100% from a heat conductance between the gas input lumen and the gas exhaust lumen per unit length averaged along all of the shaft length, for any subsection having a length equal to 20% of the shaft length.

In a preferred embodiment the gas input lumen and the gas exhaust lumen are substantially coaxial throughout their length, and the probe further comprises a spacing agent for maintaining a distance between a wall of the gas input lumen and a wall of the gas exhaust lumen.

In an alternative preferred embodiment the gas input lumen is physically fixed with respect to the gas exhaust lumen only at the cooling tip.

Preferably, an outer diameter of the cryoprobe is less than 1.5 mm.

Preferably, the system further comprises a compressor operable to compress gas exhausting from the gas exhaust lumen.

According to further features in preferred embodiments of the invention described below, the cryoprobe comprises a first portion insertable in a body, and the first portion is substantially uniformly flexible along all its length. Preferably the cryoprobe is sufficiently flexible to be non-destructively bent more than 180°.

According to yet another aspect of the present invention there is provided a cryoprobe operable to cool body tissues to cryoablation temperatures, comprising a cooling head and a shaft, the shaft comprises a gas input lumen and a gas exhaust lumen, and thermal conduction between the gas input lumen and the gas exhaust lumen differs by no more than 30% between equal-length portions of the shaft.

According to a further aspect of the present invention there is provided a cryoprobe comprising a treatment head operable to cool body tissues to cryoablation temperatures and a shaft characterized by substantially uniform flexibility along its length, and which is sufficiently flexible to be non-destructively bent more than 180°.

According to yet a further aspect of the present invention there is provided a cryoprobe sufficiently flexible to be non-destructively bent by more than 90°.

The present invention successfully addresses the shortcomings of the presently known configurations by providing a cryoprobe of highly reduced diameter operable to cool tissues to cryoablation temperatures.

The present invention further successfully addresses the shortcomings of the presently known configurations by providing a cryoprobe operable to provide temperatures lower than those typically provided by cryoprobes known to prior art.

The present invention further successfully addresses the shortcomings of the presently known configurations by providing a thin and highly flexible cryoprobe operable to extend for long distances, e.g. through blood vessels and/or ducts and/or other body conduits, and to perform cryoablation at a distant site without damaging the conduit through which it passes.

The present invention further successfully addresses the shortcomings of the presently known configurations by providing a Joule-Thomson cooling system operable to achieve low temperatures within a very short lead time.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a chart showing behavior of selected gases undergoing isenthalpic expansion from high pressure and room temperature to atmospheric pressure;

FIGS. 2a and 2b are simplified schematics showing side and transverse cross sections respectively of a closed thin cryoprobe, according to an embodiment of the present invention; and

FIG. 3 is a simplified schematic of a thin open cryoprobe, according to an embodiment of the present invention.

### DESCRIPTION OF THE PREFERRED EMBODIMENTS

The present invention is of a rapid-response Joule-Thomson cooling system, and in particular of thin and flexible cryoprobes operable to cool tissues to cryoablation temperatures by expansion of high-pressure krypton and without requiring a heat exchanger between input and output gas conduits to achieve those temperatures. Specifically, the present invention can be used to enable cryocooling and cryoablation of tissues in clinical contexts wherein narrow probe diameters, high flexibility, and rapid response are desirable. The present invention can also be used to provide rapid-response cryocooling in non-medical contexts.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrases “heat-exchanging configuration” and “heat exchanger” are both used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, and xenon, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The term “ablation volume”, as used herein, is the volume of tissue which has been cooled to ablation temperatures by one or more cryoprobes.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

It is expected that during the life of this patent many relevant cryoprobes and other thermal treatment probes will be developed, and the scope of the term “cryoprobe” is intended to include all such new technologies a priori.

In discussion of the various figures described hereinbelow, like numbers refer to like parts.

In a variety of actual and potential cryosurgical applications it would be highly desirable to reduce the diameter of the cryoprobe used. As a partial but indicative list of surgical areas where availability of a highly miniaturized cryoprobe would facilitate cryosurgical procedures or indeed enable cryosurgical treatments which are not currently practical using the relatively bulky cryosurgical needles known to prior art, we note the potential for improved treatment of non-allergic rhinitis, sinusitis, parathyroid adenoma, brain tumors, spinal tumors (neuromas) and peripheral neuromas, spinal analgesia, and various treatments in opthalmology.

Thus, for example, treatment of non-allergic rhinitis and sinusitis require passing through the nose and performing accurate ablations in order to avoid undesired injury to the cartilages. In treatment of parathyroid adenoma, percutaneous US/MRI guided needle insertion through the skin of the neck, as well as the small size of the parathyroid adenoma (10-20 mm×5-7 mm), require a short and very thin needle producing a small iceball to avoid injury of the recurrent laryngeal nerve, blood vessels or trachea. In spinal analgesia, in e.g. the lumbar spine, needle diameter should be smaller than 18 G in order to be able to pass between the vertebrae. In treatment of spinal tumors and peripheral neuromas a micro needle is required for accessibility and for small ablation areas. (It should be noted that nerves may require long and deep freezing, without enlargement of the iceball.)

In a variety of actual and potential cryosurgical applications it would further be highly desirable to deploy a cryosurgery apparatus which is narrow, long, and highly flexible. For example, transitional cell carcinoma e.g. of the ureters and renal calices might be treated by a long flexible cryoprobe extended from a cystoscope inserted in a urethra. Carcinoma of the head of the pancreas might be treated by trans-ERCP cryoablation. Lung cancer might be treated with such a cryoprobe, utilizing trans-bronchoscopic cryotreatment. For cardiology and peripheral vascular occlusion (stenosis/restenosis), a catheter like flexible probe is preferable.

In preferred embodiments of the present invention, these and other clinical objectives are achieved by (inter alia) selecting for use a cooling gas which is commercially available at reasonable cost and which has the lowest boiling point (among candidate coolants), and which is capable of being liquefied in the operating tip of a cryoprobe without aid of a counter-flow heat exchanger.

The present invention is particularly useful in providing highly miniaturized embodiments of cryoprobes. In a highly miniaturized probe, the amount of surface contact between probe and tissue is necessarily at a minimum. Boiling of a liquid coolant is an efficient way to absorb heat from tissue despite the limited sized of the cryoprobe operating tip. In contrast to flow of cold gas, which has limited capacity to remove heat from the body of the probe (and hence from tissues) by heating the flowing gas, boiling of liquid removes heat by absorbing energy needed for the liquid-to-gas phase transition, while liquid temperature remains at the boiling temperature of the substance used.

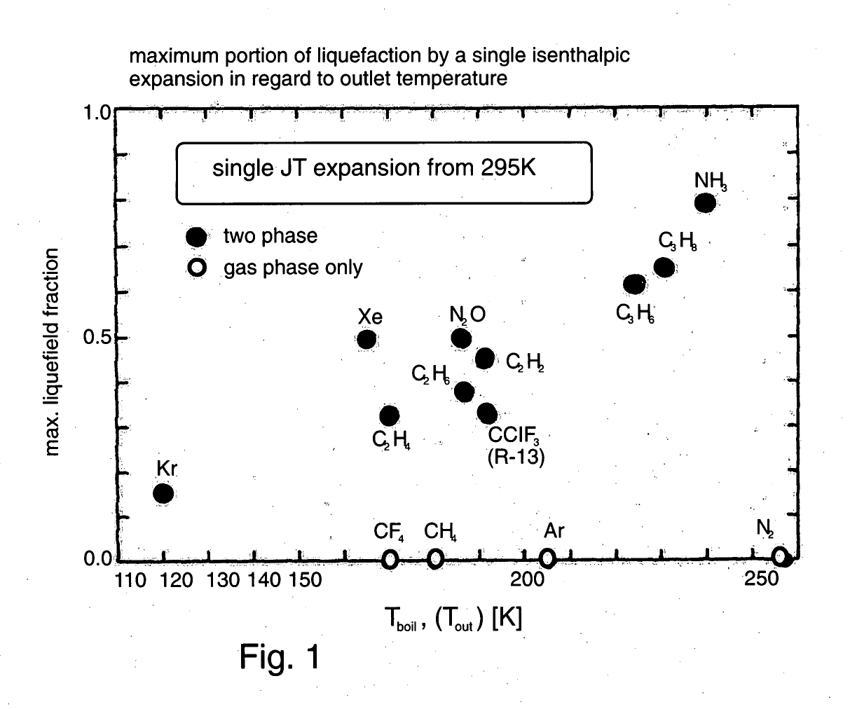

Attention is now drawn to FIG. 1, which is a chart showing behavior of selected gases undergoing isenthalpic expansion from high pressure and room temperature to atmospheric pressure. The chart of FIG. 1 is reproduced from an analysis of cooling gas characteristics to be found in: “The noble gases as favorite coolants for Joule-Thomson cryocooling”; by Maytal, Ben-Zion; published in Advances in Cryogenic Engineering, Vol. 39, pp. 1935-1939. That analysis shows that monatomic gases are advantageous candidates to be used as coolants for cryocoolers driven by Joule-Thomson cooling.

FIG. 1 summarizes behavior of various gases undergoing isenthalpic expansion from high pressure and room temperature to atmospheric pressure.

The gases CF₄, CH₄, Ar and N₂, depicted by open circles on the horizontal axis (Max liquefaction fraction=0), do not liquefy in single expansion. Minimal attainable gas temperature (Tₒᵤₜ) for these gases is, as shown, 170K, 180K, 205K and 255K respectively.

Other gases, marked by full circles, undergo liquidation during Joule-Thomson expansion, and form a jet of a mixture of gas and liquid droplets at the substance's liquid boiling temperature T_{boil}. The maximum attainable fraction of liquid in the mixture can be ascertained from the vertical axis.

Krypton is the gas of the lowest boiling point that liquefies just by a single expansion (at constant enthalpy) starting at room temperature and without a counter-flow heat exchanger pre-cooling the gas. Thus, while the monatomic gas argon does not liquefy when expanded from room temperature, and thus requires a heat exchanger to cool the gas before expansion to achieve liquefaction, the next and heavier monatomic gas on the scale of boiling points is krypton. Krypton, under Joule-Thomson expansion, can reach its liquefaction temperature without a heat exchanger.

Thus, as shown by FIG. 1, krypton is the gas of the lowest boiling point that liquefies by a single expansion starting at room temperature without using a heat exchanger. The boiling point (liquefaction temperature) of krypton is 120 K at atmospheric pressure. The maximum liquefaction fraction is ˜15%. (In practice, the liquefaction fraction depends on the exact initial conditions, including inlet and outlet pressures and initial gas temperature.)

In comparison, Xenon (Xe) reaches its boiling point at 165K, and has a maximum liquefaction fraction of 50%.

The following table, Table 1, lists the yield of liquefaction as a result of a single expansion of Krypton starting at temperatures of 295K and of 300K, from a pressure P, down to the ambient pressure of 0.1 MPa. The yield of liquefaction is the fraction of the flow that is liquefied as a result of the expansion. This yield is dependent on the applied pressure. The expanded gas reaches the boiling point at inlet pressures, which stay above 20 MPa.

TABLE 1

| P [MPa] | 300 K | 295 K |
|---|---|---|
| 15 | 0 | 0 |
| 20 | 0 | 0 |
| 25 | 0.0128 | 0.0394 |
| 30 | 0.0432 | 0.0685 |
| 35 | 0.0608 | 0.0849 |
| 40 | 0.0705 | 0.0937 |
| 45 | 0.0748 | 0.0973 |
| 50 | 0.0755 | 0.1003 |
| 55 | 0.0735 | 0.0950 |
| 60 | 0.0697 | 0.0905 |
| 65 | 0.0642 | 0.0847 |
| 70 | 0.0575 | 0.0778 |
| 90 | 0.0232 | 0.0427 |

The pressure of the expanding gas that maximizes the liquefied fraction is the Joule-Thomson inversion pressure associated with the initial temperature of the expanding stream. The state (P, T) of inversion is the one of vanishing Joule-Thomson coefficient,

$${( \frac{\partial T}{\partial P} )}_{h} = 0$$

where h is the enthalpy. For any T this condition sets the pressure P which maximizes the liquefied fraction.

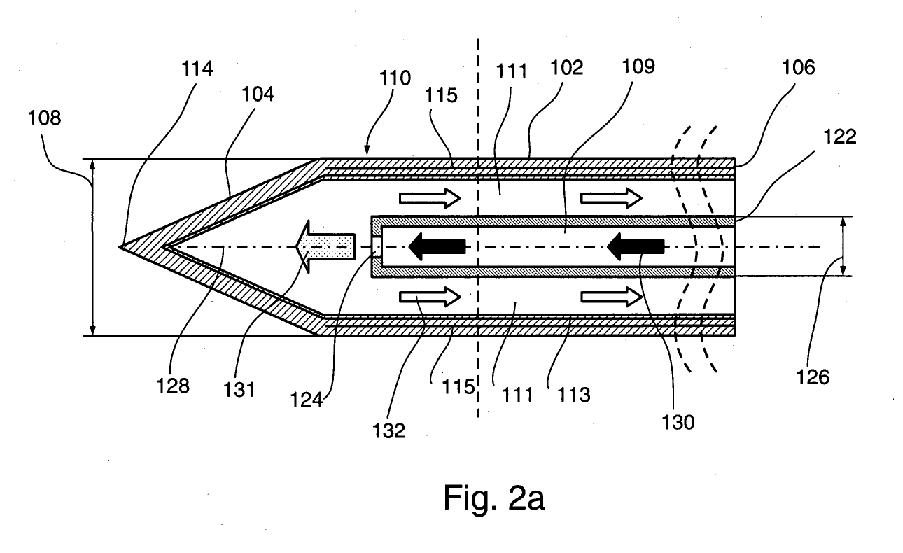

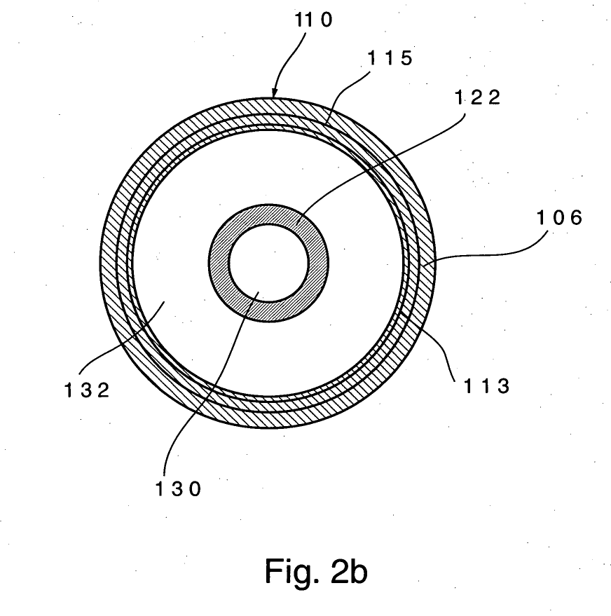

Attention is now drawn to FIGS. 2a and 2b, which are simplified schematics showing side and transverse cross sections respectively of a closed thin cryoprobe, according to an embodiment of the present invention.

FIGS. 2a and 2b present a cryoprobe 110 operable to cool to cryoablation temperatures when operated with krypton as cooling gas. Cryoprobe 110 does not comprise and does not need a heat-exchanger.

Probe 110 comprises a shaft 102 with a closed cooling tip 104 at its distal end. Cooling tip 104 is optionally configured with a sharp penetrating point 114 for penetrating biological tissue.

Shaft 102 comprises an outer tube 106 having outer diameter 108. Preferably outer diameter 108 is less than 1.5 mm, for example 0.9 mm or less.

An inner tube 122 having outer diameter 126 is positioned inside outer tube 106. Preferably outer diameter of the inner tube is less than 1.0 mm, for example 0.3 mm or less. Inner tube 122 comprises gas input lumen 109 which terminates in expansion orifice 124 at its distal end.

Space between inner tube 122 and outer tube 106 constitutes a gas exhaust lumen 111. Optionally, a mechanical spacer (not shown in these Figures) is used for holding inner tube 122 in position with respect to outer tube 106. For example, a thin wire may be loosely wound around inner tube 122 before it is inserted into outer tube 106 to maintain inner tube 122 approximately centered within outer tube 106. Alternatively, intermittent spacers may be attached to inner tube 122 before it is inserted into outer tube 106 to keep inner tube 122 approximately centered within outer tube 106. If wire or spacers are used, they must be positioned in a manner which does not significantly impede flow of gas in gas exhaust lumen 109 situated between outer tube 106 and inner tube 122.

Alternatively, inner tube 122 may be left free to move inside outer tube 106, with the two tubes joined, if at all, only at cooling tip 104, which arrangement significantly enhances flexibility of shaft 106.

In operation, krypton gas at high pressure is supplied at the proximal end of inner tube 122. High-pressure gas flow 130, marked in full arrows, transits gas input lumen 109 and exits through expansion orifice 124 into expansion chamber 128, and there forms a cold jet of gas and liquid droplet mixture 131, marked in the Figure by doted arrow.

In operation, outer walls of chamber 128 are in contact with an object to be cooled, for example biological tissue constituting a cryoablation target. Outer walls of chamber 128 thus absorb thermal energy from that object, cooling it. Thus, for example, in cryoablation treatment, probe 110 is inserted into body tissue at a cryoablation target, such as a malignant tumor. High-pressure krypton is then supplied at gas input lumen 109, causing cooling tip 104 to cool, thereby freezing target tissue and destroying diseased cells. Tissue heat absorbed by cooling tip 104 causes evaporation of liquid droplets touching the inner walls of chamber 128, and that evaporation contributes to maintaining low temperature of tip 104.

Expanded gas and evaporated liquid, marked by open arrows as low-pressure gas return 132, exit chamber 128 through annular conduit 111 formed between outer tube 106 and inner tube 122.

A heavy dash line in FIG. 2a marks the location of a cross section shown in FIG. 2b. Tubes are preferably made of metal, for example stainless steel.

In a closed-tip probe as shown in FIGS. 2a and 2b, some counter flow heat exchanging occurs as a result of the common surface of contact between gas input lumen 109 and gas exhaust lumen 111. Therefore, liquefaction temperatures may be obtained even pressure even lower than 20 Mpa, and the obtained liquefaction fraction may be greater than listed in table 1. However, it is important to note that the structural configuration presented in FIG. 2a is free of any space-consuming or flow-impeding or flexibility-limiting structures intended to enhance heat exchange between input gasses and exhaust gasses, as such enhancement is unnecessary for operation of probe 110. The configuration presented in FIG. 2a is particularly designed for use with Krypton gas, and, in contrast to prior art probes designed to achieve cryoablation temperatures using Joule-Thomson cooling, inflexible and/or space consuming heat-exchanging configurations such as the porous matrix heat exchangers and coiled heat exchangers have here been eliminated. Elimination of these heat-exchange-enhancing structures enables miniaturization of probe 110, and use of Krypton gas with probe 110 enables efficient cooling to cryoablation temperatures despite absence of the heat-exchange-enhancing configurations known to prior art.

Absence of rigid or semi-rigid heat exchangers also enables a high degree of flexibility of probe 110. As mentioned above, in a preferred alternative construction, inner tube 122 may be entirely free to move within outer tube 106. That is, inner tube 122 is not necessarily centered within outer tube 106 in this preferred embodiment. Allowing free movement of inner tube 122 within outer tube 106 further enhances flexibility of probe 110, which thus consists of a flexible inner tube positioned freely within a flexible outer tube 106, and preferably joined only at distal cooling tip 104. Probe 110 is thus rendered extremely flexible as compared to cryoprobes of prior art which incorporate rigid or semi-rigid heat exchangers in proximity to their cooling tips. In this respect it is also to be noted that although some prior art probes comprise heat exchangers positioned proximally (e.g. within a probe handle) rather than distally and near a cooling tip, the cooling efficiency of such probes is limited as the distance between heat exchanger and cooling tip increases. In contrast, since probe 110 requires no heat exchanger, cooling efficiency of probe 110 is substantially unaffected by probe length. Thus, it should be noted that although for simplicity of FIGS. 2a and 2b probe 110 is presented in the Figures as a short probe, it is to be understood that shaft 102 of probe 110 may be extremely long, thin, and flexible, enabling probe 110 to be used in context which require extending probe 110 for long distances into a body. Indeed, in a preferred method of use, probe 110 may be introduced into a body through a body conduit such as a duct, a bronchial tube, or a blood vessel, and advanced therein until treatment head 104 is in proximity to a treatment target. Thus, in an exemplary method of use, probe 110 may be advanced through the working channel of an endoscope into, say, a bladder, guided from there into a ureter, and advanced through the ureter into a kidney, there to perform cryoablation of a designated target.

It is noted that presence of cold exhaust gasses in exhaust gas lumen 111 of probe 110 will tend to cool external portions of shaft 102. Accordingly, shaft 102 is preferably provided with a heat-insulating layer 113 or an electrical shaft heating element 115. Since heat-insulating layer 113 would add thickness to probe 110 and reduce flexibility of shaft 102, heating element 115, which may be made flexible and of small dimensions, is generally to be preferred. Use of heating element 115 during cooling of treatment head 104 of probe 110 serves to protect tissues positioned in proximity to shaft 102 from damage that might otherwise be caused by inadvertent cooling of those tissues by cold gasses exhausting through gas exhaust lumen 111.

It is noted that whereas expanded gas will generally be released to the atmosphere, alternatively, expanded gas may be collected for re-use. Optionally a compressor may be supplied for compressing collected expanded gas. In a further optional construction, gas so compressed may be cooled and recycled for reuse with probe 110.

It is noted that it is a characteristic of probe 110 that gas input lumen 109 and gas exhaust lumen 111 are of substantially same length. In any case, in a preferred embodiment, lengths of lumens 109 and 111 will differ by less than 5%.

It is also noted that in a preferred embodiment, probe 110 is characterized by the fact that heat conductance along the lengths of lumens 109 and 111 is substantially uniform, given the absence of a heat-exchanging configuration similar to those used in the prior art cryoprobes discussed in the background section hereinabove and well known in the art. Heat conductance per unit length between lumens 109 and 111 being uniform, it is a characteristic of a preferred embodiment of probe 110 that measure of heat conductance between lumens 109 and 111 per unit length measured along a subsection of shaft 106 will differ only slightly, and in any case by less than 100%, from a measure of heat conductance per unit length between lumens 109 and 111 averaged over the entire length of shaft 106, for any subsection having a length equal to, say, 20% of the length of shaft 106. Similarly, in a preferred embodiment of probe 110 heat conductance between gas input lumen 109 and gas exhaust lumen 111 as measured along any two equal-length portions of shaft 106 will differ by less than 30%.

It is also noted that in a preferred embodiment, probe 110 is of substantially uniform flexibility along all its length. In an alternative embodiment probe 110 may be provided with a non-flexible section within a non-insertable portion of probe 110, yet probe 110 will in any case comprise an insertable portion which is insertable into a body of a patient, which insertable portion will be of substantially uniform flexibility along its length. Flexibility of shaft 106 may be very great. Indeed, in a preferred embodiment, shaft 106, rather like a piece of wire, may be non-destructively bent by 90°, 180°, 360°, or more, along its length.

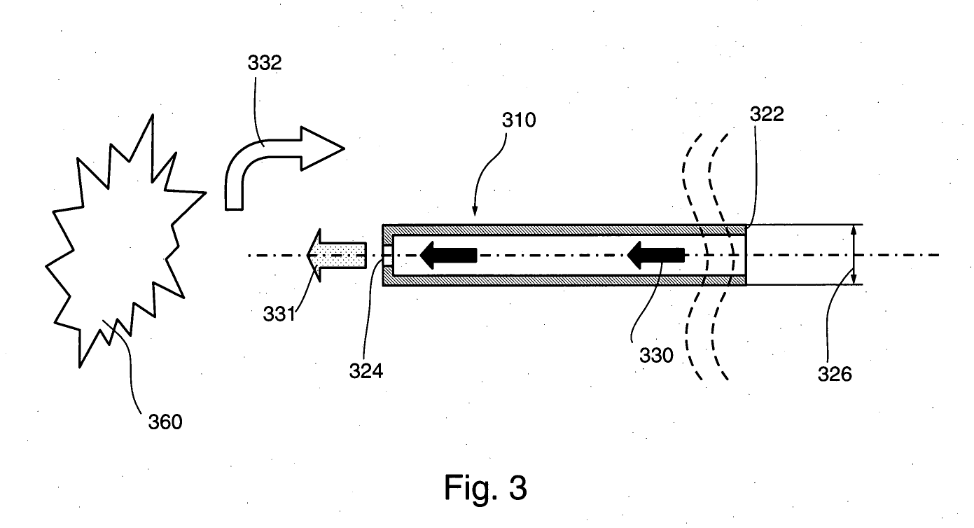

Attention is now drawn to FIG. 3, which is a simplified schematic of a thin open cryoprobe 310, according to an embodiment of the present invention.

In similarity to probe 110, cryoprobe 310 is also preferably operated using pressurized krypton as cryogen. Cryoprobe 310 comprises a gas tube 322, preferably thin, terminating in an expansion orifice 324 at its distal end. Probe 310 is preferably connected to a high-pressure krypton gas supply at its proximal end. (Of course, probes 110 and 310 may be connected to a gas supply operable to alternatively supply a cooling gas and a heating gas, according to methods well known in the art.)

Outer diameter 326 of gas tube 322 is preferably less than 1.0 mm, and more preferably 0.3 mm or less.

In operation, krypton gas at high pressure is supplied at the proximal end of gas tube 322. High-pressure gas flow 330, marked in full arrows, exits through expansion orifice 324. As explained hereinabove, high-pressure krypton, when depressurized, forms a cold jet of a mixture of gas and liquid droplets, labeled 331 in the Figure.

Jet 331 is directed at an object 360 to be cooled. Object 360 may be, for example, a biological lesion designated as a cryoablation target. Exposure of object 360 to jet 331 and evaporation of the liquid component of jet 331 from the surface of object 360 absorbs thermal energy from object 360, cooling it. In cryoablation treatment, probe 310 may be directed at an organ or it may be inserted into a natural or man-made cavity in the body and directed at target tissue such as a benign or malignant tumor. Expanded gas and evaporated liquid marked by open arrows 332 leaves the vicinity of object 360 as low-pressure gas.

Probe 310 is useable in situations where expanded gas 332 can be vented. For example, it may be used on exposed tissue such as the skin or during open surgery. In endoscopic surgery, a natural or made cavity is often inflated by gas in order to allow illumination and visual inspection of the treated organ and to make room for the surgical instruments. Gas delivery and venting means generally used to maintain a desired pressure needed for inflating the body cavity may be utilized for appropriately venting expanded gas supplied by cryoprobe 310.

The inner diameter of tube 322 and the pressure of incoming gas together determine the flow rate of the gas, and consequently the rate of heat absorption. For example, an inner diameter of 0.2 mm and an outer diameter of 0.3 mm are preferred dimensions for certain applications.

Cryoprobe 310 as described herein releases a cold jet 331 at a temperature of about 120 K. This is the coldest jet attainable by any coolant undergoing Joule-Thomson expansion from room temperature and without use of heat exchanger.

It should be appreciated that xenon may replace the krypton gas for use in probes 110 and 310. However, xenon is more expensive than krypton, and does not cool as well. The next noble gas, radon, is not generally available, and its radioactivity would make it a poor choice as cooling gas.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A cryoprobe system comprising:
(a) a gas supply operable to supply high-pressure krypton gas; and
(b) a flexible gas conduit attachable at a proximate end to said gas supply
and having an orifice positioned at a distal end of said conduit, said system being characterized in that when high-pressure krypton gas is supplied by said high-pressure cooling gas supply to said conduit, a mixture of cold, low pressure gas and liquid droplets forms outside said orifice.

2. The system of claim 1, wherein said mixture forms at a temperature inferior to 125 K.

3. The system of claim 1, wherein an outer diameter of said conduit is less than 1.5 mm.

4. A cryoprobe system comprising
(a) a supply of high-pressure krypton; and
(b) a cryoprobe which comprises
(i) a cooling tip which comprises an expansion chamber;
(ii) a shaft which comprises
(a) a gas exhaust lumen operable to exhaust gas from said cooling tip; and
(b) a gas input lumen operable to receive high-pressure krypton supplied by said high-pressure krypton supply and having a distal orifice operable to permit passage of krypton from said gas input lumen to said expansion chamber.

5. The system of claim 4, absent a portion designed as a heat exchanger serving to facilitate exchange of heat between said gas input lumen and said gas exhaust lumen.

6. The system of claim 4, operable to form a mixture of cold krypton gas and liquefied krypton droplets when krypton supplied by said gas supply traverses said orifice and enters said expansion chamber.

7. The system of claim 4, wherein said gas input lumen is positioned within said gas exhaust lumen.

8. The system of claim 4, wherein length of said gas input lumen differs from length of said gas exhaust lumen by less than 5%.

9. The system of claim 4, wherein heat conductance between said gas input lumen and said gas exhaust lumen per unit length along a subsection of said shaft differs by not more than 100% from a heat conductance between said gas input lumen and said gas exhaust lumen per unit length averaged along all of said shaft length, for any subsection having a length equal to 20% of said shaft length.

10. The system of claim 7, wherein said gas input lumen and said gas exhaust lumen are substantially coaxial throughout their length.

11. The system of claim 4, wherein said gas input lumen is physically fixed with respect to said gas exhaust lumen only at said cooling tip.

12. The system of claim 7, further comprising a spacing agent for maintaining a distance between a wall of said gas input lumen and a wall of said gas exhaust lumen.

13. The system of claim 4, wherein an outer diameter of said cryoprobe is less than 1.5 mm.

14. The system of claim 4, further comprising a compressor operable to compress gas exhausting from said gas exhaust lumen.

15. The system of claim 4, wherein said cryoprobe comprises a first portion insertable in a body, and said first portion is substantially uniformly flexible along all its length.

16. The system of claim 4, wherein said cryoprobe is sufficiently flexible to be non-destructively bent more than 180°.

17. A cryoprobe operable to cool body tissues to cryoablation temperatures, comprising a cooling head and a shaft, said shaft comprises a gas input lumen and a gas exhaust lumen, and thermal conduction between said gas input lumen and said gas exhaust lumen differs by no more than 30% between equal-length portions of said shaft.

18. A cryoprobe comprising a treatment head operable to cool body tissues to cryoablation temperatures and a shaft characterized by substantially uniform flexibility along its length.

19. The cryoprobe of claim 18, sufficiently flexible to be bent more than 180°.

20. A cryoprobe sufficiently flexible to be non-destructively bent by more than 90°.
